(19) United States
(12) Patent Application Publication
(10) Pub. No.: US 2023/0313318 A1
(43) Pub. Date: Oct. 5, 2023

(54) MICROSATELLITE GENOTYPING AND METHOD OF USE THEREOF

(71) Applicant: TRS Labs Inc, Athens, GA (US)

(72) Inventors: Crystal FRICKS, Athens, GA (US); Ray Kaplan, Grenada (WF); John W McCall, Athens, GA (US); Richard Shimkets, Athens, GA (US)

(73) Assignee: TRS Labs Inc, Athens, GA (US)

(21) Appl. No.: 18/103,141

(22) Filed: Jan. 30, 2023

Related U.S. Application Data
(60) Provisional application No. 63/327,207, filed on Apr. 4, 2022.

(51) Int. Cl. C12Q 1/6888

(52) U.S. Cl. CPC C12Q 1/6888; C12Q 1/6858

(57) ABSTRACT

Microsatellite markers, and method of use thereof, for differentiating isolates of D. immitis heartworms and/or other parasites. A method of testing new products for drug-susceptible and/or resistant isolates of heartworms using the microsatellite markers disclosed herein.

### CROSS-REFERENCE TO RELATED APPLICATIONS

This application claims the benefit of U.S. Provisional Application No. 63/327,207, filed on Apr. 4, 2022, which is incorporated herein by reference in its entirety.

### FIELD AND BACKGROUND

The present disclosure relates generally to microsatellites and genotyping thereof to differentiate multiple isolates of Dirofilaria immitis (D. immitis) and other parasites.

Microsatellites consist of short tandem repeats in DNA that are found throughout an organism's genome. Since the length of the repeats can vary within different isolates of the same species, microsatellites can be useful to differentiate between two or more isolates of the same species (Lasker and Ran, 2004). Microsatellite markers have been identified for Dirofilaria immitis and were used in an epidemiology study to examine the geography of resistant heartworms (Sanchez et al., 2020).

Canine heartworm (Dirofilaria immitis) is an extremely important parasite of dogs in the United States. Prevention of heartworm is based on compliant administration of macrocyclic lactone (ML) drugs, and all products have traditionally been labeled as 100% effective. However, resistance to ML drugs has emerged, with most cases being diagnosed in the Mississippi Delta region. Nevertheless, the spatial distribution and prevalence remains unknown.

In addition to the ML-resistant parasites, there is a wide variety of isolates or kinds of heartworm. CVM-FDA guidelines (Effectiveness of Anthelmintics: Specific Recommendations for Products Proposed for the Prevention of Heartworm Disease in Dogs, Guidance for Industry, Draft Guidance, Nov. 2022) require that at least two different heartworm isolates (often referred to as strains or biotypes) be used for testing of new heartworm products, and this assumes that only drug-susceptible isolates are used. However, the emergence of macrocyclic lactone (ML) resistance in heartworms requires that any new class of molecules would need to be tested against ML-resistant isolates as well. This results in greatly increasing the numbers of studies, and thus the number of hosts that must be required (i.e., dogs and/or cats). This further greatly impacts the costs that would accompany this expanded testing. Thus, there is a need in the art for current canine (and/or feline) heartworm drug testing models to be updated. This need also exists for other hosts and other parasites.

### SUMMARY

The present disclosure provides polymorphic microsatellite markers, and a method of use thereof, for differentiating at least three isolates of D. immitis in a host or an animal, such as dog. In certain embodiments, the three isolates of D. immitis are the macrocyclic lactone resistant JYD-34 heartworm, the susceptible GA III heartworm and SC-20. In certain embodiments, no more than six (6) microsatellite markers are used in the differentiation method disclosed herein. In other embodiments, more efficient differentiation method with two (2) to four (4) microsatellite markers are disclosed herein.

In certain embodiments, the present disclosure provides that the polymorphism microsatellite marker differentiates a peak at differentiated bases pairs with one or more corresponding primers that include, but are not limited to, Dims3, Dims4, Dims7, Dims16, Dims19, and Dims 25. The corresponding primers used to amplify these microsatellite markers/loci are provided in the table in the Detailed Description below.

In certain embodiments, the present disclosure provides that the microsatellite marker amplified by the corresponding primer Dims16 differentiates JYD-34 heartworms and GA III heartworms. In certain embodiments, JYD-34 heartworms show a peak at 202 base pairs with the Dims16 primer, while GA III heartworms show a peak at 208 base pairs with the Dims16 primer.

In certain embodiments, the present disclosure provides that the microsatellite marker amplified by the corresponding primer Dims3 differentiates GA III heartworms and JYD-34 heartworms. In certain embodiments, GA III heartworms show a peak at 382 base pairs with the Dims3 primer, while JYD-34 heartworms show a peak at 390 base pairs with the Dims3 primer.

In certain embodiments, the present disclosure provides that the microsatellite marker amplified by the corresponding primer Dims19 differentiates JYD-34 heartworms and GA III heartworms. In certain embodiments, JYD-34 heartworms show a peak at 168 base pairs with the Dims19 primer, while GA III heartworms show a peak at 162 base pairs with the Dims19 primer.

In certain embodiments, the present disclosure provides that the microsatellite marker amplified by the corresponding primer Dims7 and/or Dims25 are used in combination with the microsatellite marker amplified by either the Dims3, Dims16, or Dims19 primers to deduce genotype in differentiating JYD-34 heartworms and GA III heartworms.

In certain embodiments, the present disclosure further provides that the microsatellite marker amplified by the corresponding primer Dims4 differentiates SC-20 and JYD-34 heartworms. In certain embodiments, about 90% of JYD-34 heartworms show a peak at 363 base pairs with the Dims4 primer, while about 75% of SC-20 heartworms show a peak at 358 base pairs with the Dims4 primer. The present disclosure further provides a method of differentiating isolates of D. immitis and/or other parasites in a host and/or an animal using the microsatellite markers disclosed herein. In certain embodiments, the present disclosure provides a method of differentiating isolates of D. immitis comprising:
a) isolating DNAs from an animal having heartworms,
b) PCR amplifying the isolated DNAs using one or more corresponding primers of disclosed herein, and
c) identifying a particular isolate corresponding to the presence of the microsatellite markers amplified by the corresponding primers disclosed herein.

In certain embodiments, the isolates include but are not limited to JYD-34 heartworm, GA III heartworm, and SC-20.

The present disclosure further provides a method of testing a new product for treating drug-susceptible and/or resistant isolates of the heartworm using the microsatellite markers disclosed herein to determine the isolate(s) of heartworms remaining after the new product has been administered. In certain embodiments, no more than six (6), preferably, two (2) to four (4) microsatellite markers are used for determining the isolate(s) of the remaining heartworms after the new product for treating heartworm is administered.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

The present disclosure provides microsatellite markers, and a method of use thereof, for differentiating at least two isolates of D. immitis, such as macrocyclic lactone resistant JYD-34 heartworm and the susceptible GA Ill heartworm, in addition to SC-20. The present disclosure further provides a method of testing a new product for drug-susceptible and/or resistant isolates of the heartworm using the microsatellite markers disclosed herein to determine isolate(s) of remaining heartworms after the product has been administered.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a short chain fatty acid,” “a carnitine derivative,” or “an adjuvant,” includes, but is not limited to, combinations of two or more such short chain fatty acids, carnitine derivatives, or adjuvants, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a short chain fatty acid refers to an amount that is sufficient to achieve the desired improvement in the property modulated by the formulation component, e.g., achieving the desired level of reduction of withdrawal symptoms. The specific level in terms of wt% in a composition required as an effective amount will depend upon a variety of factors including the amount and type of short chain fatty acid, amount and type of carnitine derivative, amount and type of pharmaceutically acceptable excipients, and disorder being treated using the disclosed compositions.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### DNA Extraction and PCR Reactions Using Selected Primers

DNA was extracted from a 3-5mm excised anterior section of male and female GA Ill worms, and male and female JYD-34 worms, using components of the Promega Wizard Genomic DNA Purification Kit. These worms had been previously harvested and stored at −20 degrees. Other techniques may be used such as using pools of microfilaria and the DNeasy Mini Spin column kit.

### PCR Reactions

PCR was then run on the DNA from the known GA Ill and JYD-34 worms using the following 17 primers (Sanchez et al., 2020):

Primer sequences of 17 polymorphic microsatellite loci for
D. immiris.

| | | Allele | Repost |
|---|---|---|---|
| Locus | Primer sequences (5′-3′) | sizes (bp) | type |
| Dims3 | F: AAGACGCGGAGGAGAAAAGC | 354-394 | tetre |
| | R: TGAACGGGGAATGGATCTGC | | |
| Dims4 | F: TAGCAATGCAAACGACCACC | 331-364 | tri |
| | R: AGCTGGAGCAAAAATCAACG | | |
| Dims5 | F: GTGAGGTGCGAGTAGAGTGG | 308-338 | penta |
| | R: ACACCACCATACACCACACC | | |
| Dims7 | F: GGTCATCTACATTATTGCCGAACC | 291-306 | tri |
| | R: CCCTGGAATGATGTCTCACCG | | |
| Dims10 | F: GTTAACTGATTGATAGTACCGGG | 369-377 | tetre |
| | R: ACCGTGAAGGACTATTATAATTAAGGG | | |
| Dims11 | F: AAGCGTAGTCAGCCTCATCC | 208-211 | tri |
| | R: AGTAGGAAAGCGAAACGGGG | | |
| Dims12 | F: GCGAGGGCCATTAAGCATCC | 240-252 | hexa |
| | R: TTTCCATCCATCCACCCAGC | | |
| Dims15 | F: CTGCAAGTAGGAAATGTGGCG | 337-352 | penta |
| | R: ATCATGTCATGTCGCATCGC | | |
| Dims16 | F: CGTGGTTAGCTGCTGATACG | 210-216 | tri |
| | R: TCATCCGTGGTTGTTGTTGC | | |
| Dims19 | F: TTTGTCTTAATATCTAACCTTCGCG | 159-171 | tri |
| | R: GCGTTGTTGGATTACCGTTCC | | |
| Dims21 | F: ATAAATGAAGGCAAGCGCGC | 321-349 | tetre |
| | R: CACTTCCAGACCTCCTCTGC | | |
| Dims25 | F: TCTAGACCCTCGTTTCCCCC | 139-163 | octa |
| | R: GGTGTTTGTAAGATGATGAAGGTAGG | | |
| D2° | F: CGAATTATTACTACTATCGCCG | 105-112 | tri |
| | R: GTTTCTTTGAGGAGGAGAAGAAGAAGAGA | | |
| A05° | F: CATTGTTGTCGTGATCGCT | 196-218 | tri |
| | R: GTTTCTTACCAACAGCAGCATTAGCA | | |
| A5° | F: TTCATTTCAAGCCACAGCAG | 194-209 | tri |
| | R: GTTTCTTGGGAATCCCAGGTGTTGTAG | | |
| E4° | F: GCTTGCACTTCGTCCTTTTC | 138-152 | di |
| | R: GTTTCTTGTATGTGTGTGTAAGGGTGTG | | |
| H4° | F: GAATACAACGCAAACCTTCC | 191-197 | tri |
| | R: GTTTCTTCTGCGCTAAACAATGCAAAA | | |

For various embodiments of the present technology, PCR was run under the following conditions: 95 degrees Celsius for 10 seconds then 31 cycles of 95 degrees Celsius for 30 seconds, 55 degrees for 60 seconds, and 72 degrees for 60 seconds and afterwards a final extension of 72 degrees Celsius for 7 minutes.

### Application of Technology

The above-described experiments, analysis and technology are building blocks in the application of the technology for various embodiments of the present disclosure. In the general sense, the experiments have shown that the embodiments of the present disclosure are able to utilize a single host for testing of multiple isolates and their response to treatment.

The embodiments may vary in detail, but in general, an exemplary embodiment includes the elements of:
(1) Placing multiple different isolates within a host,
(2) Optionally provide a treatment to the host,
(3) Extract samples of the isolates from the host,
(4) DNA extraction on the recovered isolates,
(5) PCR Reactions on the extracted DNA,
(6) Identify and differentiate the isolate by observing the DNA strand obtained via analysis of the microsatellite markers (see Example 2). The pilot data had analyzed DNA from a total of 12 worms for each isolate and how the primer Dims3 and Dims16 were able to identify and differentiate certain isolates, and
(7) Thus, allowing the efficacy of the treatment to be determined with regards to each isolate within the same host.

### Example 2

### Use of Microsatellite Markers in Differentiating JYD-34 and GA III

For the entirety of the experiments, a 3-5mm anterior section of each heartworm was transected and DNA was extracted from that piece of tissue using components of the Promega Wizard Genomic DNA Purification Kit. Primers were ordered from Integrated DNA Technologies labeled with 6_FAM fluorescein. PCR was performed with 31 cycles of denaturing at 95° C., annealing at 55° C., and extension at 72° C., in the PCR reactions as described in Example 1. 6-FAM fluorescein labeled PCR products were sent to GENEWIZ/Azenta Life Sciences for fragment analysis on an ABI3730xl DNA analyzer using ABI DS33 Dye Set/LIZ500 Ladder. Results were analyzed with Peak Scanner 2 software from Applied Biosystems. These initial studies were conducted on known JYD-34 and GA III adult heartworms that had been previously harvested and stored in the freezer and were done to optimize the process and narrow down the number of primers to ultimately be used.

A total of twelve (12) known JYD-34 and twelve (12) known GA III heartworms were tested. For the Dims16 marker, twelve (12) out of the total twelve (12) JYD-34 heartworms had a peak at 202 base pairs and no GA III heartworms had a peak at 202 base pairs; and four (4) out of the total twelve (12) GA III heartworms had a peak at 208 base pairs and no JYD-34 heartworms had a peak at 208 base pairs. For the Dims3 marker, twelve (12) out of the total twelve (12) GA III heartworms had a peak at 382 base pairs and no JYD-34 heartworms had a peak at 382 base pairs; and three (3) out of the total twelve (12) JYD-34 heartworms had a peak at 390 base pairs, whereas no GA III heartworms had a peak there. The Dims7 and Dims25 markers also proved to be informative, though none of the peaks showed 100% consistency in each total twelve (12) heartworms. These data are presented in Table 1 below. The numbers represent the base pair size of each peak seen for the heartworm isolate and its respective primer.

Therefore, Dims3 and Dims16 were used as the primary microsatellite markers to distinguish the isolates of each heartworm. After analysing every heartworm in the in vivo study (described in below Example 4) with those two microsatellite markers, if the isolate of a worm could not be distinguished, fresh DNA was extracted from the worm(s) (using another 3-5 mm anterior snip) and PCR was run again with primers for the Dims3 and Dims16 markers, along with primers for the Dims7and Dims25 markers to further differentiate.

TABLE 1

| Dims3 | | | Dims7 | | Dims16 | | Dims25 | |
|---|---|---|---|---|---|---|---|---|
| GAIII | | | GAIII | | GAIII | | GAIII | |
| 370 | 382 | | 290 | 296 | 205 | 208 | 138 | 147 |
| | 382 | 386 | | 296 | 205 | 208 | | 147 |
| 370 | 382 | | 290 | 296 | 205 | | | 147 |
| 370 | 382 | | | 296 | 205 | | | 147 |
| | 382 | 386 | | 296 | 205 | | | 147 |
| 370 | 382 | 386 | 290 | 296 | 205 | | | 147 |
| 370 | 382 | | 290 | 296 | 205 | 208 | 139 | 147 |
| | 382 | 386 | | 296 | 205 | | | 147 |
| | 382 | | 290 | | 205 | | 139 | 147 |
| | 382 | | | 296 | 205 | 208 | | 147 |
| 370 | 382 | | 290 | | 205 | | 139 | 147 |
| | 382 | 386 | | 296 | 205 | | 141 | 147 |
| Dims3 | | | Dims7 | | Dims16 | | Dims25 | |
| JYD34 | | | JYD34 | | JYD34 | | JYD34 | |
| 370 | | | 293 | 296 | 202 | 205 | | 164 |
| 370 | | | | 296 | 202 | 205 | | 164 |
| 370 | 386 | | 293 | | 202 | 205 | 147 | 164 |
| 370 | | 390 | | 296 | 202 | 205 | | 164 |
| 370 | | | | 296 | 202 | 205 | | 164 |
| 370 | | 390 | 293 | | 202 | 205 | 147 | |
| 370 | | 390 | | 296 | 202 | 205 | 147 | 164 |
| 370 | | | 293 | 296 | 202 | 205 | | |
| 370 | | | | 296 | 202 | 205 | | |
| 370 | | | | 296 | 202 | | 147 | 164 |
| 370 | | | | 296 | 202 | | | 164 |
| 370 | | | 293 | 296 | 202 | | 147 | |

An in vivo study of treated and untreated dogs was conducted, and a total of 479 heartworms from the 20 dogs (See Example 4) were analysed and the results of microsatellite genotyping of these heartworms are presented in Table 2. Only seven worms were left unidentified. All three dogs in Group 1 had 100% JYD-34 heartworms and all seven dogs in Group 4 (the milbemycin treated group) had 100% JYD-34 heartworms. In the Group 2 dogs, two of the three dogs had 100% GA III heartworms. The third dog had 18 out of 20 GA III heartworms, but the other 2 heartworms were identified as having the alleles present that are most commonly seen in JYD 34 worms. Heartworms from Group 3 (infected with both isolates) were indeed differentiated as both isolates. The seven undetermined heartworms were all from Group 3. The undetermined heartworms either had peaks for the markers with specific primers for both isolates of heartworms (double positive), or there were no defining peaks for the markers with the primers present for that worm.

TABLE 2
Results of Microsatellite Genotyping

| | | | Total | | | | | |
|---|---|---|---|---|---|---|---|---|
| | | | Number of | Number of | Number of | Percentage | Number of | Percentage |
| Animal | | | Worms | Worms | JYD-34 | of JYD-34 | GA III | of GA III |
| ID | Group | Isolate | Tested | Undetermined | Worms | Worms | Worms | Worms |
| 351 | 1 | JYD34 | 18 | | 18 | 100% | 0 | 0% |
| 269 | 1 | JYD34 | 18 | | 18 | 100% | 0 | 0% |
| 267 | 1 | JYD34 | 18 | | 18 | 100% | 0 | 0% |
| 270 | 2 | GAIII | 20 | | 2 | 10% | 18 | 90% |
| 273 | 2 | GAIII | 18 | | 0 | 0% | 18 | 100% |
| 278 | 2 | GAIII | 14 | | 0 | 0% | 14 | 100% |
| 265 | 3 | JYD34 + GAIII | 33 | 3 | 13 | 39% | 17 | 52% |
| 264 | 3 | JYD34 + GAIII | 30 | 1 | 18 | 60% | 11 | 37% |
| 357 | 3 | JYD34 + GAIII | 42 | 1 | 23 | 55% | 18 | 43% |
| 262 | 3 | JYD34 + GAIII | 34 | | 20 | 59% | 14 | 41% |
| 279 | 3 | JYD34 + GAIII | 42 | | 24 | 57% | 18 | 43% |
| 284 | 3 | JYD34 + GAIII | 32 | 1 | 17 | 53% | 13 | 41% |
| 282 | 3 | JYD34 + GAIII | 29 | 1 | 14 | 48% | 13 | 45% |
| 277 | 4 | JYD34 + GAIII + MBO | 18 | | 18 | 100% | 0 | 0% |
| 263 | 4 | JYD34 + GAIII + MBO | 15 | | 15 | 100% | 0 | 0% |
| 283 | 4 | JYD34 + GAIII + MBO | 21 | | 21 | 100% | 0 | 0% |
| 271 | 4 | JYD34 + GAIII + MBO | 22 | | 22 | 100% | 0 | 0% |
| 276 | 4 | JYD34 + GAIII + MBO | 14 | | 14 | 100% | 0 | 0% |
| 280 | 4 | JYD34 + GAIII + MBO | 21 | | 21 | 100% | 0 | 0% |
| 275 | 4 | JYD34 + GAIII + MBO | 20 | | 20 | 100% | 0 | 0% |

Therefore, the studies in this Example demonstrate the use of a few microsatellite markers, with primers such as Dims3, Dims7, Dims16 and/or Dims25 to differentiate at least two isolates of D. immitis and accurately determined which heartworms were harvested from each infected dog. The primers and their peaks used to differentiate between GA Ill and JYD-34 is provided in Table 3 below. The Dims19 primer was discovered as useful after the completion of the in vivo study (see Example 4).

TABLE 3
Primers and their peaks used to differentiate
between GA III and JYD-34

| Strain | Dims3 | Dims16 | Dims19 | Dims7* | Dims25* |
|---|---|---|---|---|---|
| GA III | 382 | 208 | 162 | 290 | 138 |
| JYD-34 | 390 | 202 | 168 | 293 | 164 |

*Dims7 and Dims 25 can be used as backup or confirmation, but they are not as exclusive as the other primers corresponding to their peaks.

### Example 3

### Use of Microsatellite Markers in Differentiating JYD-34, GA III and SC-20

After completing and submitting the PCR products for all worms, additional data became available from GENEWIZ/Azenta Life Sciences. Two plates that had previously been sent for analysis were later completed. The PCR products in these two plates were to look at another D. immitis isolate, SC-20, and to further examine the Dims19 primer.

For the Dims19 primer, all of the GA III worms analyzed (n=6) had a peak at 162, whereas the JYD-34 worms did not. All of the JYD-34 worms analyzed (n=6) had a peak at 168, whereas the GA III did not. This primer could be used to genotype the worms that are not identified with other primers.

In addition, the preliminary results suggest that the Dims4 primer could be used to identify the SC-20 isolate when compared to JYD-34 and GA III.

After receiving these additional data, the extracted DNA of 24 known GA III worms and 24 known JYD-34 worms was amplified with the Dims19 primer. The results are summarized in Table 4, which shows that the Dims19 primer is a useful marker in differentiating between the GA III and JYD-34 isolates, as well. With data from a larger data pool of knowns, this primer could be used as the sole primer for distinguishing between these two isolates.

TABLE 4
Additional Data for Dims19 Primer

| | Worms Positive for Peak | Worms Positive for Peak |
|---|---|---|
| Strain of Worm | (Allele) at 162 bp | (Allele) at 168 bp |
| GA III | 23/24 | 1/24 |
| JYD-34 | 0/24 | 24/24 |

Further, extracted DNA from 20 known SC-20 worms, 20 known GA III worms, and 20 known JYD-34 worms were amplified using the Dims4 primer. Results are summarized in Tablet 6 below.

TABLE 5
Data for the Dims4 Primer

| | Worms Positive | Worms Positive | Worms Positive |
|---|---|---|---|
| | for Peak (Allele) | for Peak (Allele) | for Peak (Allele) |
| Strain of Worm | at 352 | at 358 | at 363 |
| SC-20 | 1/20 | 15/20 | 0/20 |
| GA III | 12/20 | 4/20 | 5/20 |
| JYD-34 | 1/20 | 0/20 | 18/20 |

Though these results may not as definitive as the results for the Dims19 primer for differentiating between GA III and JYD-34, the Dims4 primer could be useful to differentiate between SC-20 and JYD-34 as no SC-20 worms had a peak at 363, whereas 90% of the JYD-34 worms did, and no JYD-34 worms had a peak at 358, whereas 75% of the SC-20 worms did.

### Example 4

### Brief Description of In Vivo Study Design

Twenty adult Beagles dogs, both male and female, were used in this study. Before the study began, physical exams of each adult dog were completed, and blood was drawn to screen for microfilaria and D. immitis antigen, all of which were negative. 30-days before the treatment study day, dogs were subcutaneously injected with third stage of infective larvae of JYD-34, GA III, or both (see Table 6 below). On the treatment study day, dogs in group 4 were treated with a commercially available oral medication containing milbemycin oxime. Heartworm antigen tests were performed again on the 90^{th} day from the treatment study day, all of which were negative. On the 124^{th} day from the treatment study day, dogs were humanely euthanized, and adult heartworms were recovered from the heart and lungs. Worms from each dog were individually placed in wells of labelled 24 well plates and stored at −20 degrees Celsius until evaluation. Personnel in charge of recovering worms and genotyping were blinded to the groups.

TABLE 6
Study Groups

| | | | MBO | | | |
|---|---|---|---|---|---|---|
| | D. immitis | Trt. | Dosage | | Approximate | Total No. |
| Group | Isolate | Group | (mg/kg) | Route | Trt. Day | Animals |
| 1 | JYD-34 | Untreated | 0 | NA | 0 | 3 |
| | | Control | | | | |
| | | Animals | | | | |
| 2 | GA III | Untreated | 0 | NA | 0 | 3 |
| | | Control | | | | |
| | | Animals | | | | |
| 3 | JYD-34 + | Untreated | 0 | NA | 0 | 7 |
| | GA III | Control | | | | |
| | | Animals | | | | |
| 4 | JYD-34 + | Treated | 500-999 μg/kg | Oral | 0 | 7 |
| | GA III | Animals | | | | |

MBO = Milbemycin oxime,
Trt. = Treatment;
No. = Number;
NA = Not Applicable

In summary, the Examples provided herein demonstrate that microsatellite markers can be used to differentiate isolates of D. immitis when dogs (or other animals) are infected by different isolates. Though statistical analysis could solidify the results, it is still clear to see that microsatellites can be used to differentiate isolates of D. immitis when different isolates are used to infect one dog. Depending on the level of confidence required, between 1-6 primers can be used to distinguish isolates of D. immitis. The use of this technology could significantly cut down the number of dogs used for heartworm research and/or to other parasites or hosts of D. immitis that present with multiple isolates.

### References

Lasker B A, Ran Y. Analysis of polymorphic microsatellite markers for typing Penicillium marneffei isolates. J Clin Microbiol. 2004 April; 42(4):1483-90. doi: 10.1128/JCMA2.4.1483-1490.2004. PMID: 15070993; PMCID: PMC387604.

Sanchez, J., G. Dharmarajan, M. M. George, C. Pulaski, A. J. Wolstenholme, J. S. Gilleard, R. M. Kaplan. 2020. Using population genetics to examine relationships of Dirofilaria immitis based on both macrocyclic lactone-resistance status and geography. Vet. Parasitol. 283, 109125.

Effectiveness of Anthelmintics: Specific Recommendations for Products Proposed for the Prevention of Heartworm Disease in Dogs, Guidance for Industry, Draft Guidance, Nov. 2022.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A microsatellite marker for differentiating one or more isolates of D. immitis in an animal, said microsatellite marker differentiates a peak at differentiated bases pairs with one or more corresponding primers.

2. The microsatellite marker of claim 1, wherein the corresponding primer is selected from the group consisting of Dims3, Dims16, Dims7, Dims19, Dims25 and Dims4.

3. The microsatellite marker of claim 1, wherein the isolate is selected from the group consisting of JYD-34 heartworm, GA III heartworm and SC-20.

4. The microsatellite marker of claim 1, wherein the corresponding primer is Dims16 primer that differentiates JYD-34 heartworms and GA III heartworms.

5. The microsatellite marker of claim 4, wherein JYD-34 heartworms show a peak at 202 base pairs with the Dims16 primer.

6. The microsatellite marker of claim 5, wherein GA III heartworms show a peak at 208 base pairs with the Dims16 primer.

7. The microsatellite marker of claim 1, wherein the corresponding primer is Dims3 primer that differentiates GA III heartworm and JYD-34 heartworms.

8. The microsatellite marker of claim 7, wherein GA III heartworms show a peak at 382 base pairs with the Dims3 primer.

9. The microsatellite marker of claim 8, wherein JYD-34 heartworms show a peak at 390 base pairs with the Dims3 primer.

10. The microsatellite marker of claim 1, wherein the corresponding primer is Dims19 primer that differentiates JYD-34 heartworms and GA III heartworms.

11. The microsatellite marker of claim 10, wherein JYD-34 heartworms show a peak at 168 base pairs with the Dims19 primer.

12. The microsatellite marker of claim 11, wherein GA III heartworms show a peak at 162 base pairs with the Dims19 primer.

13. The microsatellite marker of claim 2, wherein the corresponding primer is Dims7 and/or Dims25 that are used in combination with Dims 3, Dims16, or Dims19 primers to deduce genotype in differentiating JYD-34 heartworms and GA III heartworms.

14. The microsatellite marker of claim 1, wherein the corresponding primer is Dims4 primer that differentiates SC-20 and JYD-34 heartworms.

15. The microsatellite marker of claim 14, wherein about 90% of JYD-34 heartworms show a peak at 363 base pairs with the Dims4 primer.

16. The microsatellite marker of claim 15, wherein about 75% of SC-20 heartworms show a peak at 358 base pairs with the Dims4 primer.

17. The microsatellite marker of claim 1, wherein the animal is a dog or a host for D. immitis.

18. A method of differentiating one or more isolates of heartworms in an animal, comprising:
c) isolating DNAs from the animal having heartworms,
d) PCR amplifying the isolated DNAs using one or more corresponding primers of claim 1, and
c) identifying a particular isolate corresponding to the presence of the microsatellite markers of claim 1.

19. The method of claim 18, wherein the isolate is selected from the group consisting of JYD-34 heartworm, GA III heartworm and SC-20.

20. A method of testing a new product for drug-susceptible or resistant isolate of heartworms using one or more microsatellite marker of claim 1.
